(19) United States
(12) Patent Application Publication
(10) Pub. No.: US 2004/0248959 A1
(43) Pub. Date: Dec. 9, 2004

(54) Histamine containing eye drops for visual identification of lymphoid tissue within the tissues of the eye, that are to be samples for further diagnostic testing

(76) Inventor: Scott C. Bender, Chinle, AZ (US)

Correspondence Address: Scott Bender, DVM, P.O. Box 2204, Chinle, AZ, 86503, US

(21) Appl. No.: 10/827,225

(22) Filed: Apr. 20, 2004

Related U.S. Application Data
(60) Provisional application No. 60/466,943, filed on Apr. 30, 2003.

(51) Int. Cl. A61K 31/4172

(52) U.S. Cl. 514/400

(57) ABSTRACT

Histamine containing eye drops are applied to the eye(s) of an animal or person being tested for any disease that uses lymphoid tissue from the eye for diagnosis. The Histamine containing eye drops cause a local vasodilatation that highlights lymphoid tissue within the eye helping the tester identify and sample the correct tissue, thus improving the diagnostic results. These Histamine eye drops help improve in-vivo testing for Scrapie Disease of Sheep, and may aid in the in-vivo diagnostic testing of diseases like Chronic Wasting Disease (CWD) of Deer and Elk, Creutzfedt-Jakob (CJD) and variant Creutzfedt-Jakob (vCJD) of Humans.

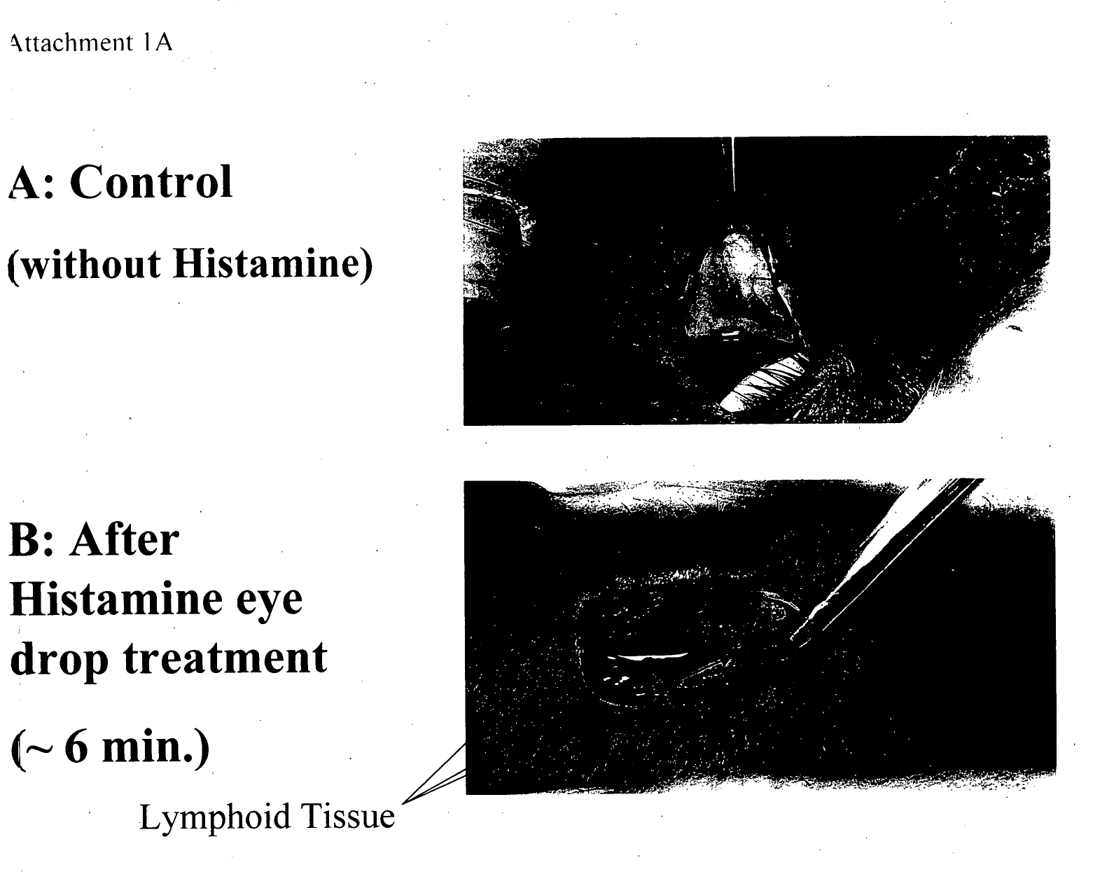

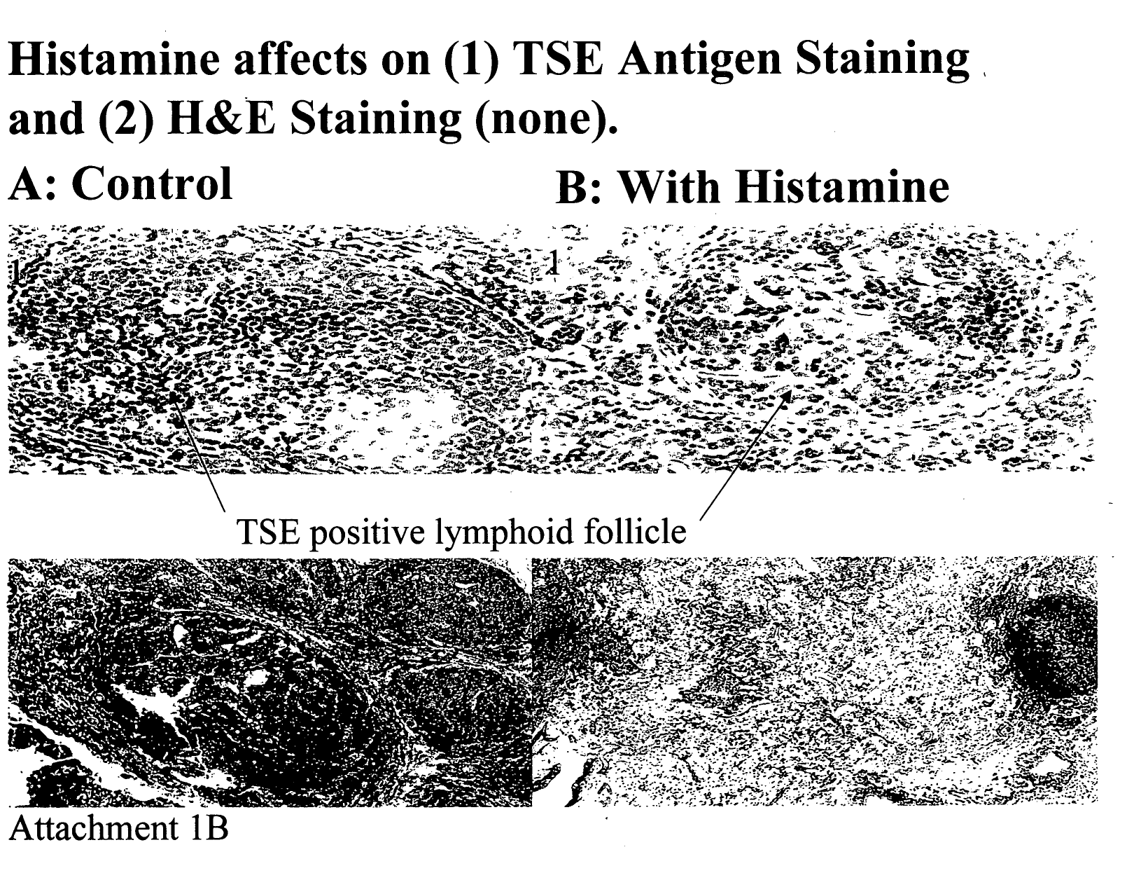

### FEDERAL RESEARCH STATEMENT

[Not applicable to this Invention]

### BACKGROUND OF INVENTION

1) Medical Field Application

2) Adjunct method for collecting tissue for medical diagnostic testing.

3) Improves visual identification of lymphoid tissue within the eye, that is collected for diagnostic disease testing, thus improving diagnostic efficiency of the test being conducted.

### SUMMARY OF INVENTION

1) Histamine containing eye drops are applied to the eye of an animal or person being tested, to visually identify lymphoid tissue within the area of the eye. The tissue is then sampled and submitted for further diagnostic testing.

2) Examples of diseases that could potentially be tested for using this include; Sheep=Scrapie, Deer and Elk=Chronic Wasting Disease (CWD), Human=Creutzfedt-Jakob Disease (CJD), Cattle=Bovine Spongiform Encephalopathy (BSE). This is only an illustrated example, as there may be many other diseases and species that this could be used to aid diagnostic testing.

3) Prior to the use of Histamine eye drops, it has been extremely difficult to identify lymphoid tissue in the eye. This limited the success of diagnostic tests that used lymphoid tissue from the eye. By using Histamine containing eye drops, the percentage of useful and diagnostic samples is often increased from ˜40% to ˜95%.

4) Provisional Application No. 60/466,943.

### DETAILED DESCRIPTION

1) Histamine eye drops are made by adding Histamine phosphate to an aqueous solution. This solution could be sterile water, 0.5% Proparacaine Hydrochloride Ophthalmic Solution (commercially available), or other ophthalmic medications.

2) Histamine is added to make a 1% Histamine containing solution. (10 mg/ml).

3) Once the eye drops are prepared, the drops are then placed into the eye of the animal or person being tested. The lymphoid tissue within the eye can then be visually identified and sampled (Biopsy) for further testing.

4) This visual identification is based upon the Histamine receptors in the lymphoid follicles of the eye, causing a localized vasodilatation of blood vessels. The lymphoid tissue contained within the eye area is then highlighted by the increase blood flow, causing a contrast difference changing the color of the lymphoid tissue from the non lymphoid tissue surrounding it.

(See Attachment 1A).

5) Ideal sampling is done ˜6 minutes after the application of the Histamine containing eye drops.

6) The naturally occurring endogenous Histaminease contained within the eye then deactivates the externally applied Histamine, thus returning the lymphoid and non lymphoid tissues to their pre application condition.

7) The Histamine eye drops does not adversely affect the diagnostic test being used. In the attached example, the histamine did not affect TSE antigen staining or H & E Staining of a diagnostic lymphoid follicle.

(See Attachment 1B)

What I claim as my invention is:

1) Adding Histamine phosphate to eye drops making an eye drop solution for diagnostic testing (making aqueous Histamine, Histamine/Proparacaine, etc)

2) Using the Histamine containing eye drops to identify lymphoid tissue within the eye, that is sampled/biopsied for further diagnostic testing.

3) I claim: that the idea for this invention came to me while looking at the inflamed lymphoid tissue in my own eyes. Then upon taking an antihistamine medication to resolve the problem, the idea of making and using a Histamine eye drop to highlight the lymphoid tissue in the eye, would make sampling the correct (lymphoid) tissue easier. (refer to #1 above)

4) I claim: that I have used these Histamine containing eye drops in sheep for the identification of lymphoid tissue sampled for the in-vivo Scrapie diagnostic test. (refer to #2 above)

5) I claim: that the Histamine containing eye drops made my collection of diagnostic viable lymphoid tissue increase from ˜65% to ˜95%, with other diagnostic clinicians reporting similar improvements in their sampling ability. (refer to #2 above)

6) I claim: that the use of Histamine containing eye drops did not adversely affect the diagnostic test nor harm any of the animals/species it has been used in. (refer to#2 above)

7) I claim: that I have used the Histamine containing eye drops in deer and elk to identify lymphoid tissue contained within the eye of those species. This lymphoid tissue was identified and collected for in-vivo testing for the Chronic Wasting Disease (CWD) of Deer and Elk. (refer to #2 above)

8) I claim: that these Histamine containing eye drops may be useful to identify lymphoid tissue for sampling in the eyes other animal species and humans; for diseases and diagnostic tests not yet identified, invented or described above. (example: collecting lymphoid tissue for diagnostic testing for Creutzfedt-Jakob Disease (CJD) and variant Creutzfedt-Jakob Disease (vCJD) of Humans). (refer to #2 above)
